(19) United States
(12) Patent Application Publication
(10) Pub. No.: US 2016/0374594 A1
(43) Pub. Date: Dec. 29, 2016

(54) SYSTEM FOR MONITORING A DOPAMINERGIC ACTIVITY

(71) Applicant: KONINKLIJKE PHILIPS N.V., EINDHOVEN (NL)

(72) Inventors: GARY NELSON GARCIA MOLINA, MADISON, WI (US); RONALDUS MARIA AARTS, GELDROP (NL)

(21) Appl. No.: 15/194,554

(22) Filed: Jun. 27, 2016

Related U.S. Application Data
(60) Provisional application No. 62/184,991, filed on Jun. 26, 2015.

(51) Int. Cl. A61B 5/11; A61B 5/16; A61B 5/0496; A61B 5/00; A61B 3/14

(52) U.S. Cl. CPC A61B 5/1103; A61B 5/1118; A61B 5/486; A61B 3/14; A61B 5/0496; A61B 5/6803; A61B 5/4857; A61B 5/4082; A61B 5/16; A61B 5/4839

(57) ABSTRACT

The present invention relates to a system for monitoring a dopaminergic activity of a subject, wherein the system comprises a detection unit for detecting an eye blink signal relating to eye blinks of the subject; an analysis unit for determining, based on the detected eye blink signal, an eye blink feature indicative of the dopaminergic activity of the subject and for comparing the eye blink feature with a reference eye blink feature; and a feedback unit for providing feedback relating to the dopaminergic activity of the subject based on the comparison of the eye blink feature and the reference eye blink feature. Further aspects of the present invention relate to a corresponding method and computer program. The approach permits early screening of a dopaminergic activity and continuous monitoring of the patient's response to dopamine drugs, for example.

### CROSS-REFERENCE TO RELATED APPLICATIONS

The present application claims the priority benefit of U.S. Patent Application No. 62/184,991, filed Jun. 26, 2015, the entire disclosure of which is hereby incorporated herein by reference.

### FIELD OF THE INVENTION

The present invention relates to a system for monitoring a dopaminergic activity of a subject. The present invention furthermore relates to a corresponding method as well as to a corresponding computer program for carrying out said method.

### BACKGROUND

Disorders related to dysfunctional dopaminergic activity (DA) have received increasing attention in recent years due to their high prevalence and their societal and economic burden. Among the most prevalent mental disorders, caused by dysfunction in the dopamine system, are: Parkinson's disease, related to abnormally low dopaminergic activity, and schizophrenia, related to abnormally high dopaminergic activity, and certain types of depression. In the US, per-year approximately 60,000 people are diagnosed with Parkinson's disease and approximately 100,000 people are diagnosed with schizophrenia. Treatment of these conditions mostly relies on medication, namely dopamine agonists for Parkinson disease and dopamine antagonists for Schizophrenia.

Inappropriate administration of dopaminergic agents can result in serious side effects including compulsive behaviors (N. Giladi, N. Weitzman, S. Schreiber, H. Shabtai, and C. Peretz, “New onset heightened interest or drive for gambling, shopping, eating or sexual activity in patients with Parkinson's disease: the role of dopamine agonist treatment and age at motor symptoms onset,” J. Psychopharmacol., vol. 21, no. 5, pp. 501-506, July 2007), increased heart rate, cardiac output, and blood pressure (J. C. Holmes and N. O. Fowler, “Direct Cardiac Effects of Dopamine,” Circ. Res., vol. 10, no. 1, pp. 68-72, January 1962).

In current medical practice adjusting the dosage of dopaminergic agents is a tedious and time-consuming process that can involve several specialist consultations. Based on the experience of the treating physician and recommendations provided by pharmaceutical companies an initial dosage of a dopaminergic agent is provided to the patient. Over the course of several weeks, the dosage of the dopaminergic agent is adjusted depending on how the patient feels, in particular by asking the patient about an improvement or deterioration of his symptoms. Such an assessment can be highly subjective. Furthermore, the effect of dopaminergic agents can strongly depend on the eating habits of the subject, since the uptake of certain dopaminergic agents can compete e.g. with proteins in nutrition as for example in milk products. Moreover, the treatment of a patient with dopaminergic agents currently requires that the patient strictly sticks to his medication schedule and takes the medication at predefined times.

In certain disorders (restless leg syndrome), dopamine agent based treatment can even lead to augmentation, i.e., a worsening of the symptoms (M. H. Silber, “Restless Legs Syndrome,” Mayo Clin. Proc., vol. 72, no. 3, pp. 261-264, March 1997). Such a deterioration of the patient's condition can be wrongly attributed to a progression of a disease. The treating physician may come to the erroneous conclusion to increase the dose of the dopamine agent.

Furthermore, patients often only consult a physician when the disease has already progressed to an extent that the patient experiences impairments by symptoms in everyday life. However, just as for several other mental disorders, it would be critically important for successful treatment to detect the disease at an early stage. For instance people with Parkinson's disease may lose up to 80% of dopamine in their bodies before symptoms appear (T. E. Golde, “The therapeutic importance of understanding mechanisms of neuronal cell death in neurodegenerative disease,” Mol. Neurodegener., vol. 4, no. 8, pp. 1-7, January 2009). Detecting and treating schizophrenia at an early stage also improves the patients' response to treatment (I. Melle, T. K. Larsen, U. Haahr, S. Friis, J. O. Johannessen, S. Opjordsmoen, E. Simonsen, B. Rishovd, P. Vaglum, and T. McGlashan, “Reducing the Duration of Untreated First-Episode Psychosis,” Arch Gen Psychiatry, vol. 61, pp. 143-150, 2004).

US 2014/0357968 A1 discloses a method and system that utilize saccadic eye movement parameters of a subject to measure dopamine levels. A saccadic eye movement refers to a rapid angular rotation of the eyes to shift the line of sight and bring an object of interest into focus.

### SUMMARY OF THE INVENTION

It is an object of the present invention to facilitate a dose adjusting process of a dopaminergic agent and in particular to permit early screening of abnormalities in dopaminergic activity. In particular to, it would be desirable to have means to unobtrusively and continuously monitor a dopaminergic activity.

In a first aspect of the present invention a system for monitoring a dopaminergic activity of a subject is presented. The system comprises:

a detection unit for detecting an eye blink signal relating to eye blinks of the subject;

an analysis unit for determining, based on the detected eye blink signal, an eye blink feature indicative of the dopaminergic activity of the subject and for comparing the eye blink feature with a reference eye blink feature; and

a feedback unit for providing feedback relating to the dopaminergic activity of the subject based on the comparison of the eye blink feature and the reference eye blink feature.

In a further aspect of the present invention a method for monitoring a dopaminergic activity of the subject is presented. Said method comprises the steps of:

receiving an eye blink signal relating to eye blinks of the subject;

determining, based on the received eye blink signal, an eye blink feature indicative of the dopaminergic activity of the subject;

comparing the eye blink feature with a reference eye blink feature; and

providing feedback relating to the dopaminergic activity of the subject based on the comparison of the eye blink feature and the reference eye blink feature.

In further aspects of the present invention, there are provided a computer program which comprises executable program code for causing a computer to perform the steps of the method disclosed herein when said computer program is carried out on a computer, as well as a non-transitory computer-readable recording medium that stores therein a computer program product, which, when executed by a processor, causes the method disclosed herein to be performed.

Preferred embodiments of the invention are defined in the dependent claims. It shall be understood that the claimed method and computer program can have similar and/or identical preferred embodiments as the claimed system and as defined in the dependent claims.

The herein presented system and method provide a possibility to unobtrusively and continuously monitor a dopaminergic activity of a subject. Thereby, the application of therapy based on dopaminergic agents or agonists can be better tailored to the patient's needs. During treatment it is important to observe the patient's response to the therapy to adjust the dosage. In particular because of the potential adverse effects of the therapy, it is desirable to have continuous monitoring of dopamine activity throughout the therapy duration. With the proposed system, evidence-based dose adjustment can be provided.

The present system comprises three main components: (i) a sensing component which is herein denoted as detection unit, (ii) a processing unit which is herein denoted as analysis unit which analyzes and interprets the signals sensed with the detection unit, (iii) an output unit which is herein denoted as feedback unit and configured to provide feedback relating to the dopaminergic activity of the subject. The feedback can be provided to the subject whose dopaminergic activity is being monitored and/or to medical personnel, in particular to a treating physician.

An eye blink feature indicative of the dopaminergic activity of the subject is determined based on the detected eye blink signal and compared with a reference eye blink feature. An advantage is that the eye blink signal can be obtained unobtrusively by the detection unit. Based on the comparison of the measured eye blink feature and the reference eye blink feature the feedback unit provides feedback relating to the dopaminergic activity of the subject. The proposed system can thus be seen as a healthcare support system, which aids a physician in finding an appropriate treatment for the patient.

As a further advantage, the proposed system can be beneficial for early screening of neurodegenerative diseases related to DA function. Hence, advantageously, a dysfunction of the dopamine system can be identified at an earlier stage, in particular before the disease has already progressed to an extent that the patient experiences impairments by symptoms in everyday life. Since early treatment can improve the patient's response, the quality of life of the patient can be increased or at least maintained at a higher level for an extended period of time.

The detection unit may include one or more sensors for detecting an eye blink signal relating to an eye blink of the subject.

The analysis unit is preferably realized as a processor which comprises one or more program modules which are configured to determine, based on the detected eye blink signal, an eye blink feature indicative of the dopaminergic activity of the subject and for comparing the eye blink feature with a reference eye blink feature. For example, it has been found that a spontaneous eye blink rate positively correlates with central dopamine activity and thus reflects the central dopamine activity. The measured eye blink feature is compared with a reference eye blink feature as normative data. Although it may seem plausible that blinking is caused by dry or irritated eyes, it is most likely that blinking is controlled by a ‘blinking center’ of the globus pallidus of the lenticular nucleus—a body of nerve cells between the base and outer surface of the brain. It has been found that greater activation of dopaminergic pathways dopamine production in the striatum is associated with a higher rate of spontaneous eye blinking.

The feedback unit may be steered by the analysis unit and may comprise one or more actuators that shall actively give a feedback, e.g. a notification or warning signal if the analysis unit, based on a comparison of the determined eye blink feature with a reference eye blink feature, determines for example that a difference between the eye blink feature and the reference eye blink feature exceeds a predetermined threshold.

In the following, some terms which are used throughout the application, shall be shortly explained and defined:

The term “dopaminergic activity” relates to a dopamine level of the subject and can serve as an indicator of a dysfunction in the dopamine system. In particular, an abnormally low dopaminergic activity can be indicative of Parkinson's disease and an abnormally high dopaminergic activity can be indicative of schizophrenia.

An “eye blink” refers to a movement of an eyelid of the subject. The eye can be completely shut. However, the upper and lower lids are not necessarily completely shut.

An “eye blink signal” relating to eye blinks of the subject can refer to a measurement signal indicative of at least one eye blink, for example a video signal of the eye or an electro-oculogram. The eye blink signal can be a contact or non-contact measurement signal.

An “eye blink feature” as used herein can refer to a quantifiable property of the eye blink that is affected or influenced by the dopaminergic activity of the subject. For example, an eye blink feature can be a duration, amplitude, slope of the eye blink signal, etc. An eye blink feature can also refer to a relation between eye blinks such as an eye blink rate, eye blink rate variation, interval between eye blinks or variation of an interval between eye blinks.

Correspondingly, the term “reference eye blink feature” can refer to an expected eye blink feature, for example an expected eye blink rate or amplitude. In particular, the reference eye blink feature can refer to an expected value or value range of the eye blink feature for comparison with the eye blink feature as determined based on the detected eye blink signal. Hence, the reference eye blink feature serves as a basis for comparison with the determined eye blink feature.

In other words, the reference eye blink feature can relate to an expected value for a healthy subject, i.e., a baseline for healthy behavior. Hence, a deviation of this healthy baseline, either positive or negative, can be indicated by the feedback unit. For example, the feedback unit can provide feedback relating to the dopaminergic activity of the subject if the comparison yields that a difference between the measured eye blink feature and the reference eye blink feature exceeds a predetermined threshold. In a further refinement, a recommendation can be provided of how to adjust a dose of a dopaminergic agent. For example, an adjustment of the dosage scales with a difference between the eye blink feature and the reference eye blink feature. Thus, a higher dosage can be recommended if a large difference between the measured eye blink feature and the reference eye blink feature is determined. A lower dosage may be recommended if a small difference between the measured eye blink feature and the reference eye blink feature is determined.

Yet another possibility to set reference values for the eye blink feature consists in taking, for example, from published research extreme values of eye blink features such as an extreme high eye blink rate for instance from publications in schizophrenia patients and extreme low values, for instance from publications in Parkinson's disease patients. The high-low levels can then be used to detect deviations in the eye blink feature at a given time. For example, the feedback unit can provide feedback relating to the dopaminergic activity of the subject if the measured eye blink feature corresponds to a malignant reference eye blink feature, for example a low value which can be indicative of Parkinson's disease.

According to an embodiment, the eye blink feature is an eye blink rate. Correspondingly, the reference eye blink feature can be a reference eye blink rate. The eye blink rate refers to a number of eye blinks in a given amount of time, for example a number of eye blinks per minute. To increase the reliability of the determination or estimation or the eye blink rate, a sufficiently long time window, e.g. 2 minutes, can be considered. In a practical context however, it can be necessary to trade accuracy with convenience. For instance, in a system that uses video monitoring to detect the eye blinks the time window to detect them may last for few seconds only. It has been shown that a high eye blink rate can be indicative of abnormally high dopaminergic activity and can thus be an indicator of schizophrenia. A low eye blink rate can in turn be indicative of abnormally low dopaminergic activity and thus be an indicator for Parkinson's disease.

According to an embodiment, the feedback unit is configured to provide a recommendation relating to an adjustment of a dose of a dopaminergic activity agent and/or to initiate treatment. Hence, the system for monitoring a dopaminergic activity can be used in clinical decision support (CDS), wherein a recommendation is provided to the subject and/or physician. Of course the physician is free to decide on how to treat the patient. Nevertheless, the physician can make an evidence-based decision, which is supported by measurements of the proposed monitoring system. If the subject is not under treatment yet, a notification can be provided to initiate treatment. A medical check-up can thus be recommended. If the subject is already under treatment, for example, a dose adjustment can be recommended. For example, assuming that the eye blink feature is an eye blink rate and that the reference eye blink feature is a previously measured eye blink rate of the same subject, a dose adjustment can be recommended (i) to decrease DA agent/agonist if the eye blink rate has increased compared to the reference eye blink rate or (ii) increase DA agent/agonist if the eye blink rate has decreased. Hence, a closed loop therapy of disorders caused by dopaminergic activity dysfunction can be achieved. It should be noted that an adjustment of a dose can also refer to changing a frequency and/or intensity of therapy, which is not necessarily medication based, such as light therapy, as an example of non-drug dopamine enhancer treatment. Adjusting a dose can also refer to stopping the medication, for example, if the dopaminergic activity corresponds to a healthy value. Thereby, unnecessary treatment can be avoided.

According to an embodiment, the detection unit comprises a camera and/or an electrode. For the case of a camera, the eye blink signal can be a video signal. The video signal can be from at least a part of the patient's face including at least a part of one eye of the subject. The detection unit can be a camera allowing the capture of an image or video of a region of the face that includes at least an eye. The detection of eye blinks from video signals is for example reported in (S. Matsuno, N. Itakura, M. Ohyama, S. Ohi, and K. Abe, “Analysis of trends in the occurrence of eyeblinks for an eyeblink input interface,” in 2014 IEEE 2nd International Workshop on Usability and Accessibility Focused Requirements Engineering (UsARE), 2014, pp. 25-31). A camera-based approach provides great flexibility, since cameras that are already available in devices such as smartphones, tablets, computers, TV sets, webcams, etc. and can be used for obtaining the eye blink signal. Hence, the monitoring of the dopaminergic activity of the subject can, for example, take place while the subject is looking at his smartphone or working at his computer. This is particularly advantageous for home health monitoring and preventive measures in order to detect a dysfunction of the dopaminergic system at an early stage before symptoms occur. A further advantage is cost effectiveness.

Alternatively or in addition, the detection unit comprises at least one electrode. The electrode can be an electrode to capture electrical activity in and/or near an ocular region of the subject. Advantageously at least one electrode is located on the front of the head, advantageously in proximity of the eye and lid. In this embodiment, the eye blink signal relating to the eye blinks of the subject can be an electro-oculogram (EOG). An optional reference electrode can be located near the ear. In general, the reference electrode is located on an electrically neutral location. The electrically neutral notion refers to a region where the electrical signal is independent from the signal of interest. Given that the ocular activity is targeted, the ear region is electrically neutral. The nose region can also be a good candidate for referencing purposes although the ear reference should provide better signal quality.

It should be noted that it can be sufficient to capture an eye blink signal from one eye only. For example the eye blink signal can be obtained using the camera of a Google® glass-like device and/or using one or more electrodes applied to a single eye. It was found that eyelid movements from both eyes occurring e.g. during spontaneous blinks are tightly synchronized.

According to an embodiment, the detection unit is comprised in a wearable device. A wearable device can incorporate one or more electrodes to capture electrical activity in and/or near an ocular region and/or a camera allowing the capture of an image of a region of the face that includes at least an eye. Advantageously, at least the detection unit of the system for monitoring the dopaminergic activity of the subject can thus be worn by the patient. An advantage of this embodiment is that a dopaminergic activity can be monitored throughout the day and thereby provide continuous monitoring. For example, the detection unit is implemented in a head-mounted device as, for example, an eyeglass-like device such as a Google® glass-like device. It should be noted that references to Google® glass are intended to concretely illustrate the concept and do not constraint the scope to a Google® glass based embodiment.

According to a further refinement, the detection unit is comprised in a head-mounted device comprising a nasal support and/or an oral support. Advantageously, the wearable device comprises a frame, as for example known from eyeglasses, but not necessarily including an ophthalmic lens. The frame is connected to the nasal support and/or at least one, preferably two, oral supports. The detection unit can be mounted on said frame. For example, the detection unit can be a camera facing an eye region of the subject for obtaining an eye blink signal relating to eye blinks of the subject. Hence, the detection unit can be conveniently worn by the patient for continuous monitoring of a dopaminergic activity.

According to a further refinement, said nasal support and/or said oral support comprises an electrode of the detection unit. As a non-restrictive example such electrode can also be a reference electrode. Thus, the head-mounted device can comprise a nasal support and the nasal support can comprise an electrode of the detection unit for detecting the eye blink signal relating to eye blinks of the subject. In addition or in the alternative, the head-mounted device comprises an oral support comprising a reference electrode. Thereby, a synergistic effect can be provided in that an electrode of the detection unit is integrated in a nasal support and/or an oral support of the head-mounted system. Thus, in addition to providing mechanical support, a measurement electrode of the detection unit can be put in place. This also improves the handling and ease of use of the proposed system.

According to an embodiment, the analysis unit is configured to identify spontaneous eye blinks in the eye blink signal and to determine a spontaneous eye blink rate as the eye blink feature. Correspondingly, the reference eye blink feature can refer to a reference spontaneous eye blink rate. For example, spontaneous eye blinks can be distinguished from voluntary eye blinks, since voluntary eye blinks can have a longer duration and higher amplitude. A higher amplitude in this context can refer to a voltage of an electro-oculogram (EOG) from an electrode-based measurement. In particular, eyelids tend to be more tightly closed or almost completely closed in voluntary eye blinks. Correspondingly, an amplitude and duration of an eye blink can also be determined in a video signal from a camera-based measurement. It has been found that reflex blinks can be neglected in determining the spontaneous eye blink rate because of the low probability of reflex eye blinks under normal circumstances. The spontaneous eye blink rate (SEBR) can be determined or estimated as the number of identified spontaneous eye blinks per time interval. It has been found that the spontaneous eye blink rate (SEBR) positively correlates with central dopamine activity and can thus be used as a noninvasive peripheral measure of dopaminergic activity.

In an embodiment, the analysis unit is configured to identify spontaneous, voluntary and/or reflex eye blinks in the eye blink signal based on one or more signal features relating to said spontaneous, voluntary and/or reflex eye blinks respectively.

A ‘voluntary eye blink’ can refer to a closing of the eyelid that is voluntarily controlled by the user.

A ‘reflex eye blink’ can refer to an eye blink that is not voluntarily controlled by the user and can be attributed to an external stimulus. A reflex eye blink involuntarily occurs in response to the external stimulus.

A ‘spontaneous eye blink’ can be defined as an eye blink which is neither a voluntary eye blink nor a reflex eye blink.

Since signal features corresponding to spontaneous, voluntary and/or reflex eye blinks can differ between subjects, the system can be calibrated for a particular user. For example, the user can be asked to perform voluntary eye blinks, for example via the feedback unit. Reflex eye blinks can be evoked by an external stimulus. The corresponding eye blink signals are analyzed for characteristic features such as duration, amplitude, slope, etc. Additional non-voluntary and non-reflex eye blinks can be seen as spontaneous eye blinks. The duration of the eye blink is a particularly useful feature to distinguish between spontaneous and voluntary eye blinks. The duration of the latter tend to be longer (>250 milliseconds) as compared to spontaneous eye blinks. Reflex eye-blinks or startle eye blinks are on the contrary rather short (<80 milliseconds).

According to an embodiment, the system further comprises a stimulus unit for evoking a reflex eye blink. Advantageously, the stimulus unit is used in calibrating the system. For example, the stimulus unit emits a light stimulus towards the eye or can play a loud sound which then evokes a reflex blink.

According to an embodiment, the analysis unit is configured to obtain the reference eye blink feature from at least one of a previously determined eye blink feature of the subject, a historic patient database and/or a research database. The reference eye blink feature can be obtained from a plurality of different sources or based on any combination thereof. An advantage of obtaining the reference eye blink feature from a previously determined eye blink feature of the subject, i.e. based on a previous measurement of the same subject, is that a patient-specific baseline can be used. Hence, a treatment outcome or progress can be monitored and tracked. This is particularly helpful when adjusting a dose of a DA agent (agonist or antagonist) because it can be directly observed whether the adjusted treatment provides the desired effect. A first measurement of the eye blink feature can be obtained before treatment and serve as a reference eye blink feature to assess, for example, an effectiveness of the treatment. Alternatively or in addition, a historic patient database can be used such that the determined eye blink feature of the subject can be compared with historic values. Advantageously, a group with similar and/or identical properties as the subject can be selected from a historic patient population to provide the historic patient database for reference. Averaging can be applied to obtain an average reference eye blink feature. Historic patients can be clustered by age, gender and the like to obtain a reference group. An advantage of obtaining the reference eye blink feature from a research database, i.e. values reported in particular in published research, is that measurements obtained under controlled laboratory conditions are provided with high reliability. The system can comprise a memory, wherein data relating to the reference eye blink feature can be stored for comparison with the determined eye blink feature based on the detected eye blink signal of the subject. The reference eye blink feature can involve the subjects or values from healthy subjects as a baseline or values from patients diagnosed with a disease, wherein the obtained values can be indicative of the disease.

According to an embodiment, the analysis unit is configured to determine a time of the day and to compare the eye blink feature with the reference eye blink feature based on the time of the day. It has been found that an eye blink feature, such as a spontaneous eye blink rate, can show a natural variation throughout the day during the wakefulness period. For example, the spontaneous eye blink rate (SEBR) increases throughout the day. The variation of the SEBR can be directly correlated with DA variation. The fact that the SEBR changes across the day can be important because this provides contextual information that is relevant to decide whether a measured SEBR at a given time of the day deviates from the reference values. In other words, a given value of an eye blink feature can be classified as indicative of a dysfunctional dopaminergic activity in the morning, whereas the same value of the eye blink feature can be within the normal range of a healthy subject in the evening. An advantage of this embodiment is that the comparison of the eye blink feature and the reference eye blink feature is more accurate.

According to an embodiment, the analysis unit is configured to determine a circadian phase of the subject and to compare the eye blink feature with the reference eye blink feature based on the circadian phase of the subject. Advantageously, the circadian rhythm or phase of the subject is taken into account. Thereby, the comparison between the eye blink feature and the reference eye blink feature can be better tailored to the subject and more meaningful results can be provided in that the determined eye blink feature can be better compared with a reference eye blink feature as it would be expected for this particular subject in his circadian rhythm.

### BRIEF DESCRIPTION OF THE DRAWINGS

These and other aspects of the invention will be apparent from and elucidated with reference to the embodiments described hereinafter. In the following drawings:

FIG. 1 shows an embodiment of a system for monitoring a dopaminergic activity of a subject according to an aspect of the present invention

FIG. 2 shows a block diagram of a system for monitoring a dopaminergic activity of a subject according to an embodiment of the present invention;

FIG. 3 shows a flow chart of a method for monitoring a dopaminergic activity of a subject;

FIG. 4 shows an exemplary graph of a spontaneous eye blink rate versus time of the day;

FIG. 5 shows electrodes applied to an eye region of a face of the subject;

FIG. 6 shows an exemplary graph of an eye blink signal;

FIG. 7 shows a further graph of an eye blink signal;

FIG. 8 shows a graph of exemplary processing steps to detect eye blinks from a video sequence;

FIG. 9 illustrates face recognition; and

FIGS. 10A and 10B show images of an eye in opened and closed state extracted from a video sequence.

### DETAILED DESCRIPTION OF THE INVENTION

FIG. 1 schematically shows an exemplary first embodiment of a system for monitoring a dopaminergic activity of a subject according to an aspect of the present invention. The system is therein denoted in its entirety by reference numeral 1. The system 1 as shown in FIG. 1 is preferably configured as a wearable system, in particular a head-mounted system that may be worn by or attached to a subject 100.

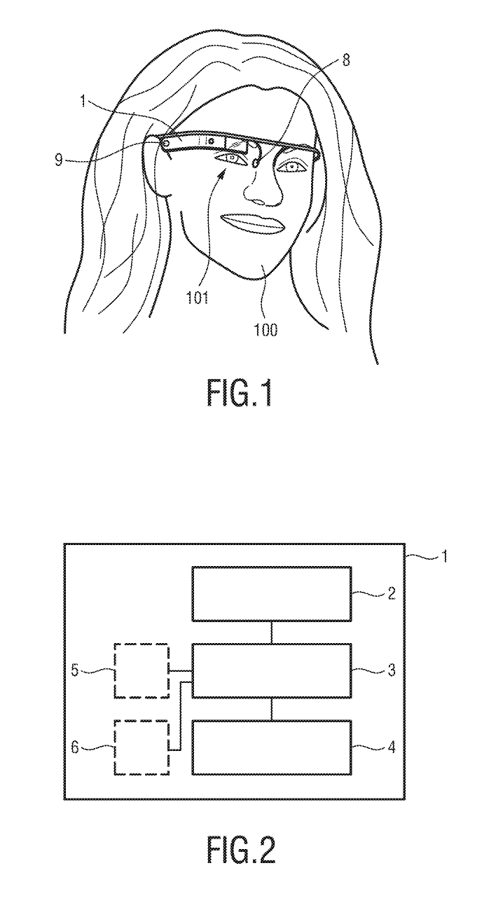

FIG. 2 shows a block diagram which schematically illustrates the components of a system for monitoring a dopaminergic activity of a subject 1 as well as their connections with each other. The system 1 for monitoring a dopaminergic activity of the subject 100 comprises a detection unit 2 for detecting an eye blink signal relating to eye blinks of the subject; an analysis unit 3 for determining, based on the detected eye blink signal, an eye blink feature indicative of the dopaminergic activity of the subject 100 and for comparing the eye blink feature with a reference eye blink feature; and a feedback unit 4 for providing feedback relating to the dopaminergic activity of the subject based on the comparison of the eye blink feature and the reference eye blink feature. The analysis unit 3 can optionally further comprise or be connected to a memory unit 5, wherein the reference eye blink feature is stored.

The detection unit 2 can comprise one or more sensors for detecting an eye blink signal relating to eye blinks of the subject. In the example illustrated in FIG. 1, the system 1 for monitoring a dopaminergic activity of the subject 100 is implemented as a head-worn device in a Google® glass-like fashion. The system in this embodiment can comprise a camera which is facing a region 101 of the right eye of the subject 100. However, the left eye or both eyes could also be used. In the shown embodiment, the eye blink signal is a video signal of the region 101 comprising the right eye. Hence, the detection unit acquires a movement of the eyelids of the subject and can determine eye blinks and eye blink features based thereon.

Alternatively or in addition, the detection unit 2 may comprise at least one electrode for contacting a facial region 101 located in proximity to the eye in particular the eyelids of the subject 100. In the shown embodiment, the eye blink signal can be an electro-oculogram relating to eye blinks of the subject 100. A synergistic effect can be provided in that the electrode of the detection unit 2 is integrated in a nasal support 8 of the head-mounted system 1. Optionally, a reference electrode for contacting a skin of the subject can further be located near an ear of the subject. Also in this case a synergistic effect can be provided in that the reference electrode is implemented in an ear support 9 of the head-mounted system 1. The reference electrode can be built into a temple tip/earpiece. Hence, the nasal support 8 and/or the oral support 9 can provide mechanical support and further be used for positioning one or more electrodes of the detection unit 2.

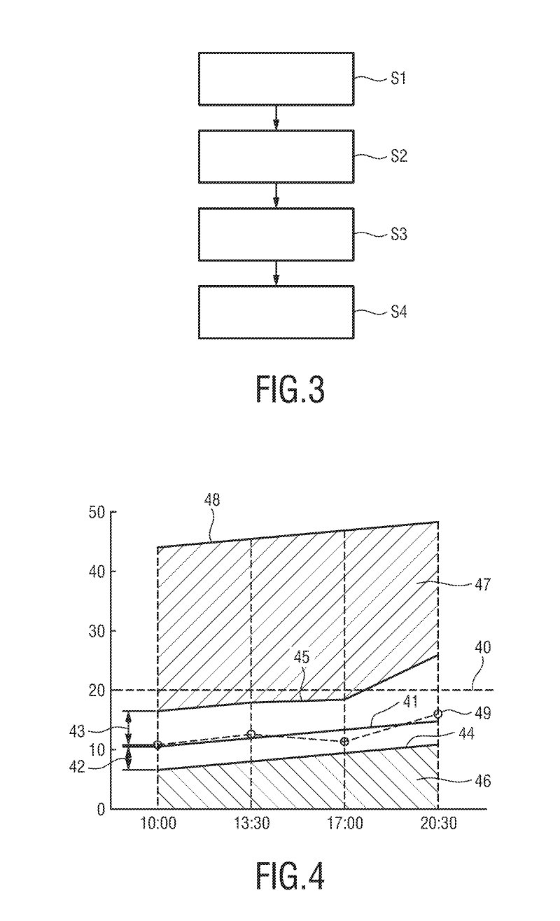

FIG. 3 shows a flow chart of a method for monitoring a dopaminergic activity of a subject, wherein the method comprises the steps of receiving an eye blink signal relating to eye blinks of the subject 100 (S1); determining, based on the received eye blink signal, an eye blink feature indicative of the dopaminergic activity of the subject 100 (S2); comparing the eye blink feature with a reference eye blink feature (S3); and providing feedback relating to the dopaminergic activity of the subject based on the comparison of the eye blink feature and the reference eye blink feature (S4).

The proposed system can assist a physician and/or the subject to be treated at different stages of disorders related to dysfunctional dopaminergic activity (DA):

Firstly, after the patient has been diagnosed with a disease related to dopaminergic activity, it is a tedious process to find the correct dose of a dopamine (agonist or antagonist) agent. Instead of having a very limited number of specialist consultations and adjusting the dosage of a dopaminergic activity agent based on perceived symptoms of the subject, the proposed system can provide continuous monitoring of the dopaminergic activity of the subject for evidence-based decision support. The corresponding measurements can be used to carefully tailor the medication to the patient's needs and may also consider the habits and lifestyle of the patient. It should further be noted that the proposed system does not necessarily require highly trained personnel for operation. Thus the economic burden regarding specialist consultations may be reduced.

Secondly, disorders related to a dysfunctional dopaminergic activity may change over time and therefore require a continuous re-adjustment of the medication. In today's practice, follow-up appointments are typically scheduled in predetermined time intervals, for example every two months. Additional appointments may be scheduled by the patient upon request, however, this often only occurs when the symptoms have already deteriorated to such an extent that the subject experiences impairments in his everyday lifestyle. When continuously monitoring a dopaminergic activity, the treatment can be adjusted at a very early stage or, alternatively, if the disease is currently going through a stable phase, unnecessary follow-up appointments can be avoided altogether.

Thirdly, another reason to continuously monitor dopaminergic activity, even when not under treatment, is to enable early detection of a DA-related mental disorder.

An exemplary embodiment of the invention will now be explained in more detail with reference to an example, wherein the eye blink feature is an eye blink rate, in particular a spontaneous eye blink rate (SEBR).

In a first step an eye blink signal relating to eye blinks of the subject is detected by the detection unit 2 of the system 1. Several options exist to measure eye blinks. A first option is electro-oculography (EOG), wherein at least one electrode 51 is attached to an eye region 102 of the subject 100 as shown in FIG. 5. Electro-oculography can be implemented through consumer systems that capture the patient's electroencephalogram (EEG). Exemplary EEG systems are commercially available, for instance by Mindo-4S (Mindo, Hsinchu, Taiwan), MindWave Mobile (Neurosky, Calif., USA), and Emotiv epoc headset (Emotiv, Eveleigh, Australia).

The detection of eye blinks is advantageously done using at least one electrode located on the front of the head and a reference electrode preferably located near the ear. An electrode that captures the eye blink signal can be referred to as active electrode. While FIG. 5 shows a band around the head that holds the electrodes 51, an implementation of the system can also take different form factors as for example eyeglasses as shown in FIG. 1.

Given that it has been found that spontaneous eye blinks manifest in a highly synchronized manner in both eyes, it is conceivable to use only one or more electrodes around a single eye for detecting an eye blink signal. Correspondingly, for example for an embodiment as depicted in FIG. 1 using a camera, it is conceivable to use only one camera to capture an eye blink signal of only one eye.

In a next step, based on the detected eye blink signal, an eye blink feature indicative of the dopaminergic activity of the subject is determined by the analysis unit 3. In a first sub-step, spontaneous eye blinks are identified in the eye blink signal by the analysis unit 3. Eye blinks can be voluntary, spontaneous or can be the result of a reflex. It has been found that a link between abnormal dopaminergic activity and blink rate can be established for spontaneous eye blinks. Detecting spontaneous eye blinks can be achieved by using features such as duration and amplitude. It has been found that during voluntary eye blinks, the eyelids tend to close almost completely.

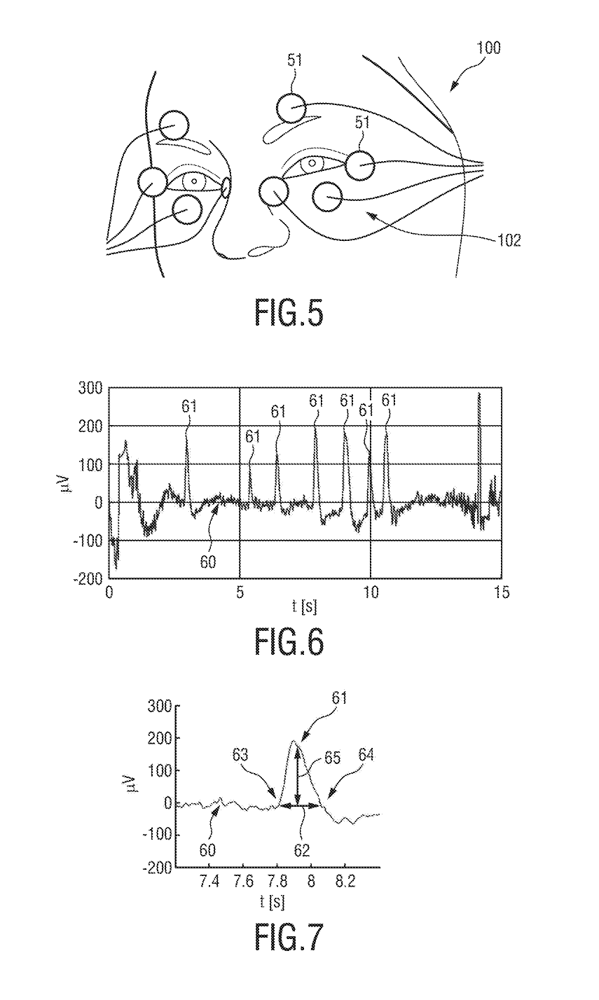

FIG. 6 and FIG. 7 show graphs of exemplary eye blink signals 60. The horizontal axis denotes a time t in seconds, whereas the vertical axis denotes the voltage of the electro-oculogram in microvolts. The eye blink signal 60 in this example is an electro-oculogram. FIG. 6 shows several peaks of the eye blink signal 60 which correspond to individual eye blinks 61.

FIG. 7 shows a peak 61 of the ocular activity in the eye blink signal 60 on a magnified time scale. Exemplary features of the eye blink signal 60 can be a duration 62 between a positive going zero crossing 63 and a negative going zero crossing 64 or an amplitude 65 of the eye blink signal 60. In the given example, the signal was rectified using e.g. a high-pass filter. The high-pass filtering correctly sets the zero-level. Eye blinks 61 in the eye blink signal 60 can be determined based on large defections of the signal, for example having an amplitude 65 larger than 70 microvolts that can be distinguished from the background activity and/or having a duration of about 200 to 300 milliseconds from positive going zero crossing zero crossing 63 to negative-going zero crossing 64. The distinction between spontaneous and voluntary eye blinks from the signal 60 in FIG. 7 can be done by taking into account that voluntary eye blinks usually last for longer, typically >250 milliseconds, and have higher amplitude because eyelids are more tightly closed.

In a next sub-step, the analysis unit 3 can be configured for estimating the eye blink rate in blinks per minute. Advantageously, the spontaneous eye blink rate (SEBR) is determined as a reliable indicator of dopaminergic activity. Optionally, a sufficiently long time window of for example two minutes can be considered in order to increase the reliability of the determination of the eye blink rate as the eye blink feature indicative of the dopaminergic activity of the subject. In a practical context however, it can be necessary to trade accuracy with convenience. Hence, a reduced measurement time may be tolerated, if a less accurate result is sufficient and if a higher convenience for the user is intended. For instance, in a system that uses video monitoring to detect the eye blinks, the time window to detect them may last for a few seconds.

Once the spontaneous eye blink rate of the subject is determined, the analysis unit can compare this eye blink feature with a reference eye blink feature, as will be described with reference to FIG. 4. The system 1 for monitoring a dopaminergic activity of the subject can further comprise a memory unit 5 for storing one or more reference eye blink features such as SEBR values, as shown in FIG. 2. The memory unit can refer to an internal or external storage or database.

FIG. 4 shows an exemplary chart of reference SEBR values for comparison with the determined SEBR value 40 by the analysis unit 3. The horizontal axis denotes the time of the day, whereas the vertical axis denotes the spontaneous eye blink rate in blinks per minute. In the shown example, an SEBR of 20 blinks per minute has been determined based on the eye blink signal 60. Advantageously, the analysis unit 3 is configured to determine a time of the day and to compare the eye blink feature with the reference eye blink feature based on the time of the day.

The comparison of the eye blink feature with the reference eye blink feature can comprise a comparison with one or more thresholds. FIG. 4 illustrates a first curve 41 which is indicative of a normal SEBR, wherein normal in this context relates to a normative, expected SEBR of an average healthy subject. If the comparison of the analysis unit 3 yields that the determined SEBR of the subject 100 differs from the normal SEBR 41 by more than a predetermined threshold the analysis unit can control the feedback unit 4 to provide feedback to the subject and/or medical personnel. The threshold can be a fixed threshold 42, 43 or can optionally be a variable threshold 44, 45 which varies depending on the time of the day and/or based on a circadian phase of the subject 100. Correspondingly, the normal SEBR 41 can be a fixed value or can vary depending on the time of the day and/or based on the circadian phase of the subject. An upper and lower threshold 42, 43 can be the same or different.

In the example shown in FIG. 4, a first region 46 between zero and the first variable predetermined threshold 44 is considered to be indicative of an abnormally low dopaminergic activity which can be an indicator of Parkinson's disease. The first region can also be referred to as low DA region. A second region 47 above the second variable predetermined threshold 45 is considered to be indicative of an abnormally high dopaminergic activity which can be an indicator for schizophrenia. The second region can also be referred to as high DA region. Thereby, the feedback unit can provide feedback relating to the dopaminergic activity of the subject based on the comparison of the eye blink feature with the reference eye blink feature.

Optionally, SEBRs above a third predetermined threshold 48 can e.g. be classified as unrealistically high, e.g. due to measurement errors or artifacts, and may thus be discarded. Optionally, an error message can be provided by the feedback unit.

The analysis unit 3 can compare the determined SEBR 40 of the subject with the SEBR 41 and determine whether the SEBR 40 of the subject differs from the normal SEBR 41 by more than a fixed predetermined threshold 42, 43 or alternatively by a variable predetermined threshold 44, 45.

As indicated in FIG. 4, the comparison can take into account the time of the day because it has been found that the SEBR is not constant throughout the duration of the whole wakefulness time. Instead of referring to an absolute time of the day, the comparison can also take the individual subject's circadian time or phase into account. Thus, the analysis unit can be configured to determine a circadian phase of the subject and to compare the determined SEBR 40 with the reference SEBR based on the circadian phase of the subject. For example, at 10 am the determined SEBR of 20 blinks per minute would be indicative of an abnormally high DA, whereas at 8:30 pm the determined SEBR would be considered to fall within a normal, healthy range between the thresholds 44 and 45.

The analysis unit can be configured to obtain the reference eye blink feature, as for example illustrated in FIG. 4, from at least one of a previously determined eye blink feature of the subject, a historic patient data base and/or a research database. For example, the normal SEBR curve 41 can be determined based on published SEBR values at different times of the day as indicated by the circles at 10 am, 1:30 pm, 5 pm and 8:30 pm in FIG. 4. A regression line can be estimated to obtain a continuum of SEBR values which spans across the whole day. The SEBR shows a stable pattern in morning, midday and afternoon hours with an average SEBR of about 11.5 blinks per minute. In addition, the SEBR shows a significant increase in evening hours up to about 15 blinks per minute. The high DA region 47 in the given example spans from the maximum normal values during daytime, indicated by curve 45, to a maximum of 44 blinks per minute as a highest reported blink rate for schizophrenia. The extrapolation over time can be obtained assuming the same regression parameters as for the normal SEBR curve 41. The low DA region 46 in the given example spans from zero to reported values for Parkinson's disease. The extrapolation over time can again be obtained assuming the same regression parameters as for the normal SEBR curve 41.

If an abnormally high or low SEBR is detected based on the comparison with the reference SEBR values and the corresponding thresholds shown in FIG. 4, then the subject and/or treating physician can be notified by the feedback unit 4 to adjust a dose, initiate treatment, or stop the medication. For example, if the subject is not under treatment yet, a recommendation for a medical check-up can be provided by the feedback unit to the subject. Thus, a recommendation can be issued if the determined eye blink feature falls within the low DA region 46 or the high DA region 47. If the subject is already under treatment and the comparison yields a determined eye blink feature in the high DA region 47 a dose adjustment can be recommended to decrease the DA agent/agonist. Correspondingly, if the subject is already under treatment and the comparison yields a determined eye blink feature in the low DA region 46 (i) a dose adjustment can be recommended to increase the DA agent/antagonist and/or to (ii) increase a frequency and/or intensity of non-drug dopamine enhancer treatment such as light therapy. An intensity of an adjustment of a dose of a dopaminergic activity agent can depend on a difference between the reference eye blink feature and the detected eye blink feature.

Optionally, for example in the case where dopaminergic therapy is continuously delivered for enhanced effect as proposed in (F. Stocchi and C. W. Olanow, “Continuous dopaminergic stimulation in early and advanced Parkinson's disease,” Neurology, vol. 62, no. 1 Suppl 1, pp. S56-S63, 2004), the monitoring of the dopaminergic activity can be used to control a drug delivery wherein the feedback unit is further configured for providing feedback for controlling a drug delivery unit for titrating the drug delivery.

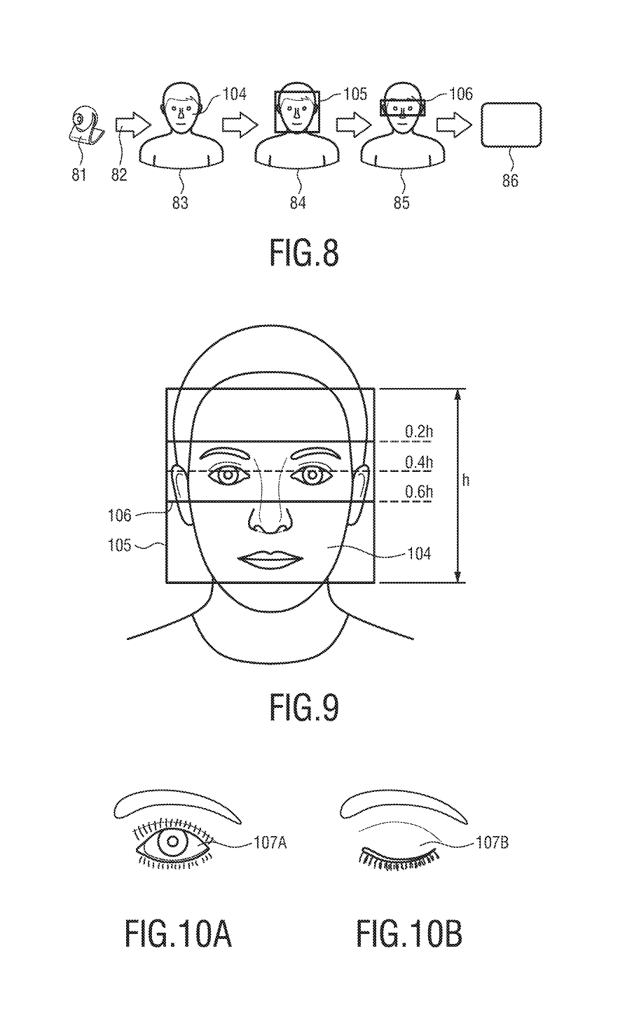

In an embodiment as described with reference to FIGS. 8 to 10B, the detection unit 2 for detecting an eye blink signal relating to eye blinks of the subject 100 is a camera 81 and the eye blink signal is a video signal 82. In this embodiment, the eye blink detection is performed using a video signal 82 from the patient's face 104. Different image acquisition units or cameras 81 can be used for example a webcam, in particular a webcam already available at a computer, a camera of a tablet computer, phone and the like or for example a Google® glass-like device, as illustrated in FIG. 1.

FIG. 8 illustrates an example of detecting eye blinks. In a first step 84, the analysis unit is configured to detect the face and provides a reduced area 105 wherein an eye region 106 with the eyes can be detected in a next step 85. Subsequently, an eyelid movement can be tracked and characterized to detect eye blinks. Based thereon, an eye blink feature indicative of the dopaminergic activity of the subject such as for example an eye blink rate can be determined in step 86. Similar to the previous embodiment based on the electro-oculogram, the distinction between spontaneous and voluntary blinks can be based on the fact that voluntary blinks are longer and thus the eyelids remain closed for a longer time period as compared to spontaneous eye blinks.

FIG. 9 illustrates the process of tracking an eye region 106 based on the reduced area 105 of the face 104 of the subject 100. The height of the reduced area 105 is given by h. The eye region 106 in this example corresponds to the region between 0.2 h and 0.6 h wherein the eyes are approximately located in the middle of the reduced region at 0.4 h.

FIGS. 10A and 10B illustrate an eye as seen with a device as illustrated in FIG. 1 in open state 107A in FIG. 10A and closed state 107B as shown in FIG. 10B.

In an embodiment, the analysis unit is configured to obtain reference eye blink features from previously determined eye blink features of the subject, for whom a dopaminergic activity is to be monitored. For example the patient's own SEBR measured at a previous point in time can serve as a reference. Thus, previously determined eye blink features in particular tracked across a longitudinal period of the same subject can be considered. An advantage of this approach is that detecting deviating eye blink features can be more accurate when having a reference period from the same subject. However, it should be considered that the subject may have already been manifesting at least early symptoms for abnormal dopaminergic activity by the time the first eye blink features have been collected. Further, comparing the currently determined eye blink feature with a previously determined eye blink feature of the same subject can be useful for the purposes of treatment-outcome monitoring where it is useful to have a patient's own eye blink feature as a reference.

While the previous non-limiting embodiments focused on evaluating a blink rate, in particular a spontaneous blink rate as the eye blink feature, it is further proposed to detect voluntary eye blink properties such as duration, amplitude, slopes and the like. A slope can refer to a ratio between a maximum amplitude reached by the eye blink and the time it takes to reach such an amplitude. Advantageously, an electro-oculogram can be evaluated. It has been found that dopamine or the lack thereof has effects on reaction time. In this embodiment, the system can be configured to establish one or more reference eye blink features for the voluntary eye blinks that can correlate with dopaminergic activity. A determined eye blink feature of a voluntary eye blink of the subject can be compared to a corresponding reference eye blink feature of a voluntary eye blink. Direct correlation between blink kinematics and dopamine have been found in the animal (Baker et al., “The Effect of Apomorphine on Blink Kinematics in Subhuman Primates with and without Facial Nerve Palsy”. Invest Ophthalmol Vis Sci. 2002 September; 43(9):2933-8). The use of voluntary eye blinks is particularly advantageous for eye blink detection based on systems, for example video-based systems, where the opportunity for detection is shorted as compared to constant monitoring approaches where patients wears for example glasses with integrated means to detect eye blinks.

In addition to, or as an alternative to voluntary eye blinks, so-called reflex eye blinks can be evoked which appear, for example, in response to startling stimuli such as a relatively loud sound or bright light. Referring to the embodiment shown in FIG. 2, the system 1 for monitoring a dopaminergic activity of a subject can thus comprise an optional stimulus unit 6 for evoking a reflex eye blink.

In conclusion, a system for monitoring a dopaminergic activity of a subject as well as a corresponding method and computer program have been proposed. The suggested approach permits early screening for abnormal dopamine activity and can enable continuous monitoring of the patient's response to dopamine drugs.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

While the invention has been illustrated and described in detail in the drawings and foregoing description, such illustration and description are to be considered illustrative or exemplary and not restrictive; the invention is not limited to the exemplary embodiments disclosed in the present disclosure. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

The present invention disclosed herein has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

Further, as one having ordinary skill in the art shall appreciate in view of the teachings provided herein, features, elements, components, etc. disclosed and described in the present disclosure/specification and/or depicted in the appended Figures may be implemented in various combinations of hardware and software, and provide functions which may be combined in a single element or multiple elements. For example, the functions of the various features, elements, components, etc. shown/illustrated/depicted in the Figures can be provided through the use of dedicated hardware as well as hardware capable of executing software in association with appropriate software. When provided by a processor, the functions can be provided by a single dedicated processor, by a single shared processor, or by a plurality of individual processors, some of which can be shared and/or multiplexed. Moreover, explicit use of the term “processor” or “controller” should not be construed to refer exclusively to hardware capable of executing software, and can implicitly include, without limitation, digital signal processor (“DSP”) hardware, memory (e.g., read only memory (“ROM”) for storing software, random access memory (“RAM”), non-volatile storage, etc.) and virtually any means and/or machine (including hardware, software, firmware, combinations thereof, etc.) which is capable of (and/or configurable) to perform and/or control a process.

Moreover, all statements herein reciting principles, aspects, and exemplary embodiments of the present invention, as well as specific examples thereof, are intended to encompass both structural and functional equivalents thereof. Additionally, it is intended that such equivalents include both currently known equivalents as well as equivalents developed in the future (e.g., any elements developed that can perform the same or substantially similar functionality, regardless of structure). Thus, for example, it will be appreciated by one having ordinary skill in the art in view of the teachings provided herein that any block diagrams presented herein can represent conceptual views of illustrative system components and/or circuitry embodying the principles of the invention. Similarly, one having ordinary skill in the art should appreciate in view of the teachings provided herein that any flow charts, flow diagrams and the like can represent various processes which can be substantially represented in computer readable storage media and so executed by a computer, processor or other device with processing capabilities, whether or not such computer or processor is explicitly shown.

Having described preferred and exemplary embodiments of a wearable device and method for collecting ocular fluid, which exemplary embodiments are intended to be illustrative and not limiting, it is noted that modifications and variations can be made by persons having ordinary skill in the art in view of the teachings provided herein, including the appended Figures and claims. It is therefore to be understood that changes can be made in/to the preferred and exemplary embodiments of the present disclosure which are within the scope of the present invention and exemplary embodiments disclosed and described herein.

Further, it is contemplated that corresponding and/or related systems incorporating and/or implementing the device or such as may be used/implemented in a device in accordance with the present disclosure are also contemplated and considered to be within the scope of the present invention. Moreover, corresponding and/or related method for manufacturing and/or using a device and/or system in accordance with the present disclosure are also contemplated and considered to be within the scope of the present invention.

What is claimed is:

1. A system for monitoring a dopaminergic activity of a subject, the system comprising:
a detection unit for detecting an eye blink signal relating to one or more eye blinks of the subject:
an analysis unit for determining, based on the detected eye blink signal, an eye blink feature indicative of the dopaminergic activity of the subject and for comparing the eye blink feature with a reference eye blink feature; and
a feedback unit for providing feedback relating to the dopaminergic activity of the subject based on the comparison of the eye blink feature and the reference eye blink feature.

2. The system according to claim 1, wherein the eye blink feature comprises an eye blink rate.

3. The system according to claim 1, wherein the feedback unit is configured to at least one of provide a recommendation relating to an adjustment of a dose of a dopaminergic activity agent or initiate treatment.

4. The system according to claim 1, wherein the detection unit comprises at least one of a camera or an electrode.

5. The system according to claim 1, wherein the detection unit is comprised in a wearable device.

6. The system according to claim 5, wherein the detection unit is comprised in a head-mounted device comprising at least one of a nasal support or an oral support.

7. The system according to claim 6, wherein the at least one nasal support or oral support comprises an electrode of the detection unit.

8. The system according to claim 1, wherein the analysis unit is configured to identify spontaneous eye blinks in the eye blink signal and to determine a spontaneous eye blink rate as the eye blink feature.

9. The system according to claim 1, wherein the analysis unit is configured to identify at least one of spontaneous, voluntary or reflex eye blinks in the eye blink signal based of one or more signal features relating to the at least one of spontaneous, voluntary or reflex eye blinks.

10. The system according to claim 1, further comprising a stimulus unit structured and configured for evoking a reflex eye blink.

11. The system according to claim 1, wherein the analysis unit is configured to obtain the reference eye blink feature from at least one of a previously determined eye blink feature of the subject, a historic patient database or a research database.

12. The system according to claim 1, wherein the analysis unit is configured to determine a time of day and to compare the eye blink feature with the reference eye blink feature based on the time of day.

13. The system according to claim 1, wherein the analysis unit is configured to determine a circadian phase of the subject and to compare the eye blink feature with the reference eye blink feature based on the circadian phase of the subject.

14. A method for monitoring a dopaminergic activity of a subject, comprising the steps of:
receiving an eye blink signal relating to one or more eye blinks of the subject;
determining, based on the received eye blink signal, an eye blink feature indicative of the dopaminergic activity of the subject;
comparing the eye blink feature with a reference eye blink feature; and providing feedback relating to the dopaminergic activity of the subject based on the comparison of the eye blink feature and the reference eye blink feature.

15. A computer program comprising executable program code structured and configured for causing a computer to carry out the steps of the method according to claim 14 when the computer program is executed on a computer.
